(19) United States
(12) Patent Application Publication
(10) Pub. No.: US 2005/0079153 A1
(43) Pub. Date: Apr. 14, 2005

(54) Methods for enhancing immune functions in neonatal mammals by administration of IL-18

(75) Inventor: Marcus E. Kehrli JR., Terre Haute, IN (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/626,826

(22) Filed: Jul. 22, 2003

Related U.S. Application Data
(60) Provisional application No. 60/403,393, filed on Aug. 14, 2002.

(51) Int. Cl. A61K 38/20

(52) U.S. Cl. 424/85.2

(57) ABSTRACT

The present invention provides methods for increasing the production of interferon-γ in neonatal mammals in response to a stimulus by administration of interleukin-1 8 to neonatal mammals. The present invention further provides methods for enhancing the immunity in neonatal mammals and protecting neonatal mammals against infectious diseases caused by protozoal, bacterial, fungal or viral pathogens.

### FIELD OF THE INVENTION

The present invention relates to methods for increasing the production of interferon-γ in young mammals in response to a stimulus by administration of interleukin-18. The methods of the present invention are useful for enhancing the function of the immune system in young mammals and protecting young mammals against infectious diseases caused by a protozoal, bacterial, fungal or viral pathogen.

### BACKGROUND OF THE INVENTION

Neonatal mammals exhibit greatly reduced levels of interferon-γ relative to older mammals (Rajaraman, V., B. J. Nonnecke, R. L. Horst. J. Dairy Sci. 80:2380-90, 1997). Mammals typically do not exhibit normal immune competence until they approach puberty (Hauser, M. A., M. D. Koob, J. A. Roth. Am. J. Vet. Res. 47:152-153, 1986). Although exactly when mature capacity for interferon-γ is reached is not known, they are likely several weeks to months old. Interferon-γ is known to be an immune modulator (Steinbeck, M. J., J. A. Roth, M. L. Kaeberle. Cell Immun. 98:137-144,1986; Bielefeldt Ohmann, H., L. A. Babiuk. J. Interferon Res. 6: 123-136, 1986; Saulnier, D., S. Martinod, B. Charley. Ann. Rech. Vet. 22:1-9, 1991) and the relative lack of interferon-γ is associated with impaired immunity (Ishikawa, H., T. Shirahata, K. Hasegawa. J. Vet. Med. Sci. 56:735-738, 1994) and increased susceptibility to serious infections. (Chiang, Y.-W., J. A. Roth, J. J. Andrews. Am. J. Vet. Res. 51:759-762, 1990; Harp, J. A., R. E. Sacco. J. Parasitol. 82:245-9, 1996; Harp, J. A., W. M. Whitmire, R. Sacco. J. Parasitol. 80:67-72, 1994; Chen, W., J. A. Harp, A. G. Harmsen. Infect. Immun. 61:3928-32, 1993; Chen, W., J. A. Harp, A. G. Harmsen, E. A. Havell. Infect. Immun. 61:3548-51, 1993)

### SUMMARY OF THE INVENTION

The present inventors have demonstrated for the first time that administration of interleukin-18 to neonatal mammals led to an increased capacity of lymphocytes from these mammals to produce interferon-γ in vitro in response to a stimulus.

Accordingly, one embodiment of the present invention provides a method for enhancing the capacity of young mammals to produce interferon-γ by administering interleukin-18 to the mammals.

In a preferred embodiment, interleukin-18 is administered to young calves to enhance the capacity of the young calves to produce interferon-γ.

Another embodiment of the present invention provides a method for protecting young mammals against infectious diseases by administering interleukin-1 8 to the mammals.

In a preferred embodiment, interleukin-18 is administered to young calves to protect the young calves against infectious diseases.

### BRIEF DESCRIPTION OF THE DRAWINGS

FIG. 1 depicts the effect of administration of recombinant bovine IL-18 to newborn calves. Each group is the mean of data from 6 calves administered injections s.i.d. for 5 days (see arrows).

### DETAILED DESCRIPTION OF THE INVENTION

One embodiment of the present invention provides a method for enhancing the capacity of young mammals to produce interferon-γ by administering interleukin-1 8 to these mammals.

By “mammals” is meant cattle, swine, horses, sheep, goats, dogs and cats.

By “young mammals” is meant mammals of an age less than three months old, preferably less than one month old. Typically, mammals less than one month old do not exhibit normal levels of interferon-γ.

By “enhancing the capacity” to produce interferon-γ is meant an increased production of interferon-γ in response to a stimulus by about 35% to about 500%, relative to the average production of interferon-γ by young control mammals of the same age which have not been administered IL-1 8. The production of interferon-γ in response to a stimulus can be measured by in vitro and ex vivo assays which have been amply described in the art and in the example that follows (Rajaraman, V., B. J. Nonnecke, R. L. Horst. J. Dairy Sci. 80:2380-90,1997).

In a preferred embodiment of the present invention, IL-18 is administered to young calves to enhance the capacity of the young calves to produce interferon-γ.

Interleukin-18 molecules suitable for use in the administration are preferably a mammalian IL-18 molecule, including human, murine, rat, bovine (Shoda, L. K., D. S. Zarlenga, A. Hirano, W. C. Brown. J Interferon Cytokine Res. 19:1169-77, 1999), porcine, equine, feline and canine IL-18. A most preferred IL-18 for administration to a mammal is the IL-18 from the same animal species as the mammal. For example, bovine IL-18 is preferred for administration to a young calf.

According to the present invention, an IL-18 molecule can be administered in the form of polypeptides, which can be readily obtained via extraction or purification from natural sources, via organic chemical synthesis, via recombinant DNA technology, or via commercial sources.

Both full-length, wild-type IL-18 proteins and functional derivatives thereof can be used in the administration. A “functional derivative” of a wild type IL-18 molecule as used herein refers to a modified IL-18 protein, which differs in amino acid sequence from wild type IL-18 by a substitution, insertion or deletion of one or more amino acid residues, but nevertheless has substantially the same activity as the wild type IL-18 in enhancing the production of interferon-γ in young mammals.

Further according to the present invention, a nucleotide sequence encoding IL-18 or a functional derivative thereof can also be used in the administration. Preferably, such nucleotide sequence is provided in an expression vector which is capable of driving the expression of IL-18 molecules in young mammals.

The expression vector can be a plasmid or viral vector such as retroviral, adenoviral and adeno-associated viral vector. Typically, an expression vector includes a promoter sequence which is operably linked to the IL-18 coding sequence. Examples of suitable promoters include (human) cytomegalovirus immediate early promoter (Seed, B. et al., Nature 329, 840-842, 1987; Fynan, E. F. et al., Proc. Natl. Acad. Sci. 90, 11478-11482, 1993; Ulmer, J. B. et al., Science 259, 1745-1748, 1993), Rous sarcoma virus LTR (RSV, Gorman, C. M. et al., Proc. Natl. Acad. Sci. 79, 6777-6781, 1982; Fynan et al., supra; Ulmer et al., supra), the MPSV LTR (Stacey at el., J. Virology 50, 725-732, 1984), SV40 immediate early promoter (Sprague J. et al., J. Virology 45, 773, 1983), the metallothionein promoter (Brinster, R. L. et al., Nature 296, 39-42,1982), the major late promoter of Ad2, the β-actin promoter (Tang et al., Nature 356, 152-154, 1992).

The expression vectors can also include other regulatory sequences, such as terminator and polyadenylation sequences. Sequences suitable for use include bovine growth hormone polyadenylation sequence, the SV40 polyadenylation sequence, and the human cytomegalovirus (hCMV) terminator and polyadenylation sequences.

The expression vectors can also include nucleotide sequences coding for other cytokines, which are appropriate for use in conjunction with IL-18 to enhance the immune function of neonatal mammals.

The IL-18 molecule, either in the form of polypeptide or in the form of an expression vector, can be administered together with other cytokines, which are appropriate for use in conjunction with IL-18 to enhance the immune function of neonatal mammals. Other cytokines which are appropriate for use in conjunction with IL-18 to enhance immune function of neonatal mammals might include but are not limited to: G-CSF, GM-CSF, IL-3, IL-7, IL-15, IL-17.

Other biologically active agents which can be administered in conjunction with IL-18 include, e.g., antiparasiticides, antibacterials, antifungals and antivirals, and the like.

According to the present invention, IL-18 and other appropriate biological agents can be admixed with a pharmaceutically acceptable carrier. Suitable pharmaceutical carriers include but are not limited to water, saline, adjuvant (such as oil emulsions, aluminium salts, derivatives of muramyl dipeptide, monophosphoryl lipid A, liposomes, QS21, MF-59, Iscoms, and the like), diluents, stabilizers (such as serum albumins, gelatins, saccharides including glucose, fructose, sucrose, maltose, lactose, trehalose, sorbitol, mannitol, maltitol, and lactitol), and buffers with phosphoric acid or succinic acid.

The compositions containing IL-18, a pharmaceutical carrier and any other appropriate biological agent may take any form that is suitable for oral, mucosal, or parenteral administration to neonatal mammals. For oral use, the compositions may be formulated as solutions, syrups, suspensions, tablets, capsules and the like. For parenteral use, the compositions according to the present invention may be formulated in a form suitable for injection such as suspensions, solutions, dispersions, emulsions, and the like. Preparation of the compositions according to the present invention is carried out by means conventional for the skilled person.

Preferred routes of administration are parenteral routes, e.g., intramuscular injection, intravenous injection, intradermal injection, subcutaneous injection, and mucosal routes, e.g. nasal drops, eye drops, (aerosol) sprays, and the like.

According to the present invention, administration of an IL-1 8-containing composition to mammals should begin shortly after birth, preferably, within one to two days after birth. The dosage and the number of times of the administration depends on the condition of the neonatal mammal (e.g., weight, response to the administration), the form of IL-18 in the composition (polypeptide or expression vector), and the route of administration. As a general rule, an IL-18-containing composition can be administered parenterally at a dose in the range of about 1 μg to 500 μg IL-18/kg per dose for about 1-4 times/day for about one to twenty-eight days. In a preferred embodiment, purified recombinant mammalian IL-18 proteins are given to neonatal mammals at 20 μg/kg/dose/day via subcutaneous injection for 5 days. A most preferred embodiment is a single injection of a dose of IL-18 in a formulation capable of providing a sustained effect of 14-28 days.

According to the present invention, administration of interleukin-18 to neonatal mammals increases the capacity of lymphocytes from these mammals to produce interferon-γ. Therefore, another embodiment of the present invention provides a method for protecting young mammals against infectious diseases by administering interleukin-18 to the mammals.

In a preferred embodiment, interleukin-18 is administered to young calves to protect the young calves against infectious diseases.

By “protecting” is meant enhancing the immunity and resistance against a disease, accelerating the recovery from a disease, or eliminating or alleviating the syndromes and symptoms of a disease.

Diseases against which the present method affords protection include, but are not limited to infectious diseases caused by a protozoal, bacterial, fungal or viral pathogen.

The type of IL-18 molecules appropriate for use in the administration, other biological active agents and pharmaceutical carriers which can be included in the administration, the schedule, dose and routes of administration are as described hereinabove.

The present invention is further illustrated by the following example.

### EXAMPLE

Test Materials:

| | | |
|---|---|---|
| 1. | Compound Name: | recombinant bovine IL-18 |
| | Dosage Form: | Injectable |
| | Potency: | 1.107 mg/mL |
| | Formulation: | Dialysis buffer |
| 2. | Compound Name: | Dialysis buffer |
| | Dosage Form: | Injectable |
| | Potency: | N/A |
| | Formulation: | 20 mM NaH₂PO₄, 500 mM NaC1, 0.1 mM |
| | | EDTA, 25% glycerol (v/v), pH 8.04 |

Animals:

| | |
|---|---|
| Species/breed: | Bovine/Holstein |
| Initial weight: | 27-57 kg |
| Sex: | Male and female |
| Origin: | Calves born on site |
| Identification: | Ear tags |
| Pre-treatment | First Defense ® bolus, ImmuCell Corp. |

Management:

| | |
|---|---|
| Housing: | Individual calf hutches on a gravel base with straw |
| | bedding. |
| Feeding and | Colostrum (5% of body weight twice daily) on day 1 & 2 |
| watering | |
| method: | |
| Environmental | Clean hutches 2x/week. |
| control: | |

Design:

| Treatment | Cytokine Dose | Day | Timing | No. Calves |
|---|---|---|---|---|
| T1 | Vehicle control | 0 | SID-X1 | 4 |
| T2 | 5 μg/kg SC | 0, 1, 2, 3, 4 | SID-X5 | 5 |
| T3 | 10 μg/kg SC | 0, 1, 2, 3, 4 | SID-X5 | 5 |
| T4 | 20 μg/kg SC | 0, 1, 2, 3, 4 | SID-X5 | 5 |

Experimental Procedure:

When a calf was born, it was given 1-2 quarts of colostrum and an oral commercial bolus of antibodies (against E. coli K99⁺ and rotavirus). Calves born before 9 AM were bled and study day zero began that calendar date for that calf. Calves born after 9 AM were processed as normal except that study day zero began the next calendar day for purposes of completing the laboratory assays with blood samples. Test article injections were given subcutaneously ahead of the prescapular region of the neck. Alternating sides of the neck were used for injections on successive days. Leukograms were determined using a Coulter counter and CD45 vs. SSC on the flow cytometer. Peripheral blood mononuclear cells were isolated over Percoll density gradients (sp. gr.=1.084) from calves and three lab control cows. Isolated cells were washed and adjusted to 4×10⁶/mL in RPMI-1640 with antibiotic/antimycotic and 100 μL added to 100 μL culture media (RMPI-1640 with 25 mM HEPES, antibiotic/antimycotic and 10% heat inactivated FBS) with or without 2 μg/mL Con-A for 44 hours. Supernatants from these cultures were harvested and interferon-γ levels determined using the Boviga™ bovine gamma interferon assay kit from CSL Veterinary adapted for a quantitative assay with recombinant bovine IFN-γ in a standard curve. Calf clinical scores were determined each morning on study days 0-7, 14 and 21. Scoring was as follows: (0)=normal, healthy; (1)=mildly ill; (2)=moderately ill; (3)=severely ill; (4)=moribund.

Data Analysis:

Assessment of test article efficacy was determined based upon comparisons of interferon-γ production between treatment groups. Data were analyzed using the MIXED procedure of PC-SAS version 6.12. The model included treatment, time and their interaction. Covariance within calves across time was modeled using the REPEATED statement with a spherical covariance structure to account for unequally spaced sampling times. Tests for significance were based upon the main treatment effect compared with the vehicle treatment group.

Results:

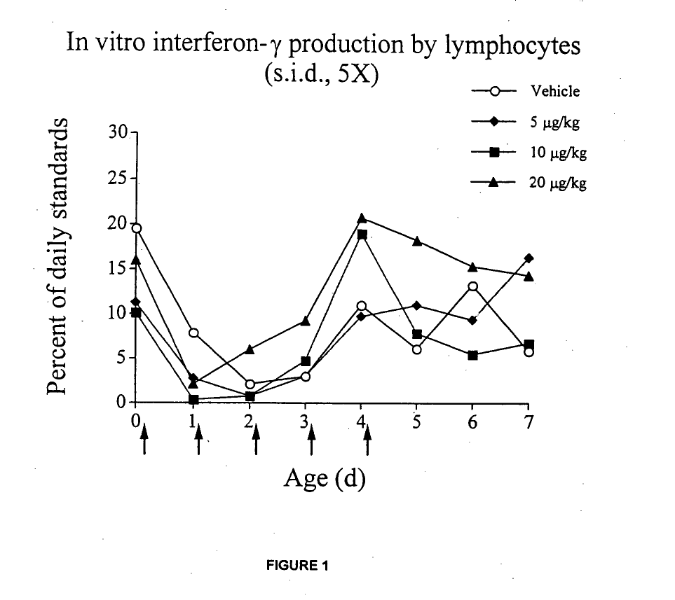

FIG. 1 summarizes the ability of isolated mononuclear cells (lymphocytes+monocytes) to produce interferon-γ in response to mitogen stimulation. Consistent with published data, the ability of calf mononuclear cells to produce interferon-γ in response to mitogen stimulation was a fraction of adult capacity shortly after birth, this capacity further declined to a nadir at 2 days of age. Calves were still at less than 25% of adult capacity at 21 days of age. Calves that received 20 μg IL-18/kg, SC, 5× had the highest observed levels of induced interferon-γ production for a 5-day period beginning 3 days after the first injection (15.6% of adult capacity versus 7.9% for controls). This represents a nearly 100% increase relative to the average production of interferon-γ by young control calves of the same age which had not been administered IL-18. For this 5-day period, the effect of increasing mononuclear cell interferon-γ production was significant at P<0.05.

Accordingly, in accordance with the present invention, administration of recombinant bovine IL-18 at 20 μg/kg, SC, 5×, increased ex vivo interferon-γ production by mitogen-stimulated mononuclear cells.

1. A method for enhancing the capacity of a young mammal to produce interferon-γ comprising administering interleukin-18 to said mammal.

2. The method of claim 1, wherein said young mammal is of an age of less than one month old.

3. The method of claim 1, wherein said interleukin-1 8 is an IL-1 8 from the same animal species as the mammal.

4. The method of claim 3, wherein said IL-18 is in the form of a recombinant protein.

5. The method of claim 3, wherein said IL-18 is in the form of a nucleotide sequence coding for said IL-18.

6. The method of claim 5, wherein said nucleotide sequence coding for said IL-18 is provided in an expression vector.

7. The method of claim 3, wherein said IL-18 is administered in conjunction with at least one other cytokine.

8. The method of claim 3, wherein said IL-18 is provided in a pharmaceutically acceptable carrier.

9. The method of claim 3, wherein said IL-18 is administered to said mammal via an oral, a mucosal, or a parenteral route.

10. The method of claim 9, wherein said parenteral route is selected from the group consisting of an intramuscular, intravenous, intradermal, and subcutaneous route.

11. The method of claim 3, wherein said IL-18 is first administered to said mammal within about two days after the birth of said mammal.

12. The method of claim 11, wherein said IL-18 is first administered to said mammal within 1 day after the birth of said mammal.

13. The method of claim 4, wherein the IL-18 protein is administered at about 1 to 500 μg/kg per dose, and one dose per day for about one to twenty-eight days.

14. The method of claim 2, wherein said young mammal is a young calf, and said IL-18 is bovine IL-18.

15. A method for protecting a young mammal against an infectious disease, comprising administering interleukin-18 to said mammal.

16. The method of claim 15, wherein said infectious disease is caused by a protozoa, bacterial, fungal or viral pathogen.

17. The method of claim 15, wherein said young mammal is of an age of less than one month old.

18. The method of claim 15, wherein said interleukin-18 is an IL-18 from the same animal species as the mammal.

19. The method of claim 18, wherein said IL-18 is in the form of a recombinant protein.

20. The method of claim 18, wherein said IL-18 is in the form of a nucleotide sequence coding for said IL-18.

21. The method of claim 20, wherein said nucleotide sequence coding for said IL-18 is provided in an expression vector.

22. The method of claim 18, wherein said IL-18 is administered in conjunction with at least one other cytokine.

23. The method of claim 18, wherein said IL-18 is provided in a pharmaceutically acceptable carrier.

24. The method of claim 18, wherein said IL-18 is administered to said mammal via an oral, a mucosal, or a parenteral route.

25. The method of claim 24, wherein said parenteral route is selected from the group consisting of an intramuscular, intravenous, intradermal, and subcutaneous route.

26. The method of claim 18, wherein said IL-18 is first administered to said mammal within about two days after the birth of said mammal.

27. The method of claim 26, wherein said IL-18 is first administered to said mammal within 1 day after the birth of said mammal.

28. The method of claim 19, wherein the IL-18 protein is administered at about 1-500 μg/kg per dose, and one dose per day for about one to twenty-eight days.

29. The method of claim 15, wherein said young mammal is a calf, and said IL-18 is bovine IL-18.
